(19) United States
(12) Patent Application Publication
(10) Pub. No.: US 2025/0164210 A1
(43) Pub. Date: May 22, 2025

(54) TOURNIQUET HOLSTER FOR A RIFLE

(71) Applicant: First Samco, Inc., Bensalem, PA (US)

(72) Inventor: Ira Block, Bensalem, PA (US)

(73) Assignee: First Samco, Inc., Bensalem, PA (US)

(21) Appl. No.: 18/952,498

(22) Filed: Nov. 19, 2024

Related U.S. Application Data
(60) Provisional application No. 63/600,918, filed on Nov. 20, 2023.

(51) Int. Cl. F41C 27/00; A61B 17/132

(52) U.S. Cl. CPC F41C 27/00; A61B 17/132

(57) ABSTRACT

A tourniquet holster is designed to attach to the underside of a rifle stock, particularly for the AR-15 platform and provides easy and rapid access to a tourniquet in emergency situations without hindering the function or operation of the rifle. The tourniquet holster includes a rigid housing formed of opposed left and right sidewalls, a rear wall, and a front wall collectively defining a sleeve channel for storing the tourniquet. Protrusion sidewalls extend forwardly from the front wall and taper to a protrusion front wall to collectively define a protrusion area for storing the elongated windlass handle of the tourniquet. The rigid housing is sized to expose a portion of the tourniquet outside forward and rearward openings of the housing to enable a user to readily grip the tourniquet for removal from the holster.

This application claims the benefit of U.S. Provisional Application No. 63/600,918 having a filing date of Nov. 20, 2023, which is incorporated by reference as if fully set forth.

### BACKGROUND

This invention relates to accessories for firearms, and specifically accessories intended for improving safety and medical accessibility for users of firearms. In particular, this invention relates to tourniquet holsters that are arranged for operational engagement with the stock of a firearm such as a rifle.

In conditions where firearms are employed, there is a risk of gunshot wounds or other serious injuries. Rapid access to life-saving tools, such as a tourniquet, can make a significant difference in the outcome. A tourniquet is a device for stopping the flow of blood through a vein or artery, typically by compressing a limb with a cord or tight bandage. Tourniquets are important first aid equipment where there is a substantial risk of injury resulting in vein or artery damage and subsequent blood loss. Weapons users may desire to have ready access to and the ability to readily deploy a tourniquet.

Known tourniquet storage devices are not entirely satisfactory for the range of applications in which they are employed. Many existing devices store tourniquets in ways that are difficult to access quickly and easily.

Many tourniquet holsters include cloth pouches that substantially envelop the tourniquet stored therein. The cloth construction provides insufficient stability when a user attempts to retrieve the tourniquet from the pouch. As a result, users may drop the tourniquet during retrieval. In tactical situations, small delays like this may result in injury or death. Further, because the pouches cover such a substantial portion of the tourniquet, users must often grip tourniquets awkwardly when removing them, resulting in even further delay. These disadvantages can reduce users' confidence in the holster, further reducing the holster's effectiveness.

Another known tourniquet holster is arranged for securement to either side of a rifle stock using suitable bungee cords or other attachment straps. During use, when shifting a rifle stock from one shoulder to the other, the tourniquet holster may become positioned between the rifle stock and the user's cheek preventing the user from maintaining his cheek firmly resting on the rifle stock for stability. Thus, it becomes necessary to remove the tourniquet holster from between the user's cheek and the rifle stock to regain this stability. Also, adjustment of the tourniquet holster in this manner prevents the holster from obstructing the user's view. This additional step costs valuable time, and under stress, can prove difficult to complete.

Thus, there exists a need for a tourniquet holster that improves upon and advances the design of known tourniquet storage devices. Examples of new and useful tourniquet holsters relevant to the needs existing in the field are discussed below.

### SUMMARY

This invention provides a solution for rifle users, particularly those using the AR-15 platform, to have immediate access to a life-saving tourniquet without compromising the functionality or safety of the rifle. In particular, this invention relates to tourniquet holsters that are arranged for operational engagement with the stock of a firearm such as a rifle. The tourniquet holster includes a rigid housing formed of opposed left and right sidewalls, a rear wall, and a front wall collectively defining a sleeve channel for storing the tourniquet. Protrusion sidewalls extend forwardly from the front wall and taper to a protrusion front wall to collectively define a protrusion area for storing the elongated windlass handle of the tourniquet. The rigid housing is sized to expose a portion of the tourniquet outside forward and rearward openings of the housing to enable a user to readily grip the tourniquet for removal from the holster.

It is to be understood that the foregoing general description and the following detailed description of the present subject matter are merely exemplary and explanatory in nature. As such, the foregoing general description and the following detailed description should not be construed to limit the scope of the appended claims in any sense.

### BRIEF DESCRIPTION OF THE DRAWINGS

The invention will be described in conjunction with the following drawings in which like reference numerals designate like elements and wherein:

FIG. 1 is a perspective view of a prior art tourniquet holster;

FIG. 2 is front view of the prior art tourniquet holster of FIG. 1 in an open position;

FIG. 3 is a rear view of the prior art tourniquet holster of FIG. 1 in the open position;

FIG. 4 is a perspective view of the prior art tourniquet holster of FIG. 1 secured to a rifle stock and shown in an open position to receive a tourniquet shown positioned therein;

FIG. 5 is a perspective view of the prior art tourniquet holster of FIG. 1 secured to a rifle stock and shown in a closed position for retaining a tourniquet therein;

FIG. 6 is a perspective view of a first embodiment tourniquet holster of the present invention;

FIG. 7 is a front view of the first embodiment tourniquet holster of FIG. 6;

FIG. 8 is a side view of the first embodiment tourniquet holster of FIG. 6;

FIG. 9 is another side view of the first embodiment tourniquet holster of FIG. 6;

FIG. 10 is a top view of the first embodiment tourniquet holster of FIG. 6;

FIG. 11 is a bottom view of the first embodiment tourniquet holster of FIG. 6;

FIG. 12 is a perspective view of the first embodiment tourniquet holster of FIG. 6 secured to a rifle stock; and,

FIG. 13 is a perspective view of the first embodiment tourniquet holster of FIG. 6 shown secured to the rifle stock and retaining a tourniquet therein.

### DETAILED DESCRIPTION

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The invention now will be described more fully hereinafter with reference to the accompanying drawings, in which embodiments of the invention are shown. This invention may however be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. As used herein, the term “and/or” includes any and all combinations of one or more of the associated listed items.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section.

It will be understood that the elements, components, regions, layers and sections depicted in the figures are not necessarily drawn to scale.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom,” “upper” or “top,” “left” or “right,” “above” or “below,” “front” or “rear,” may be used herein to describe one element's relationship to another element as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. The invention illustratively disclosed herein suitably may be practiced in the absence of any elements that are not specifically disclosed herein.

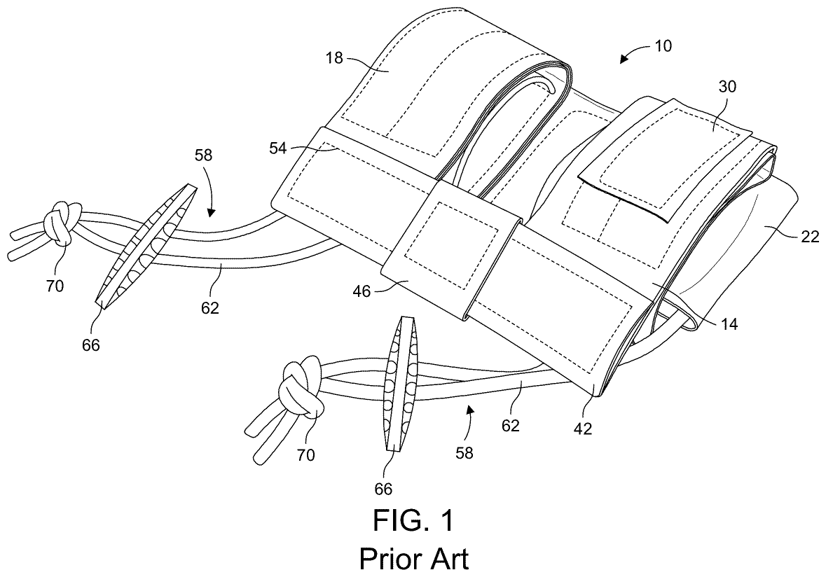

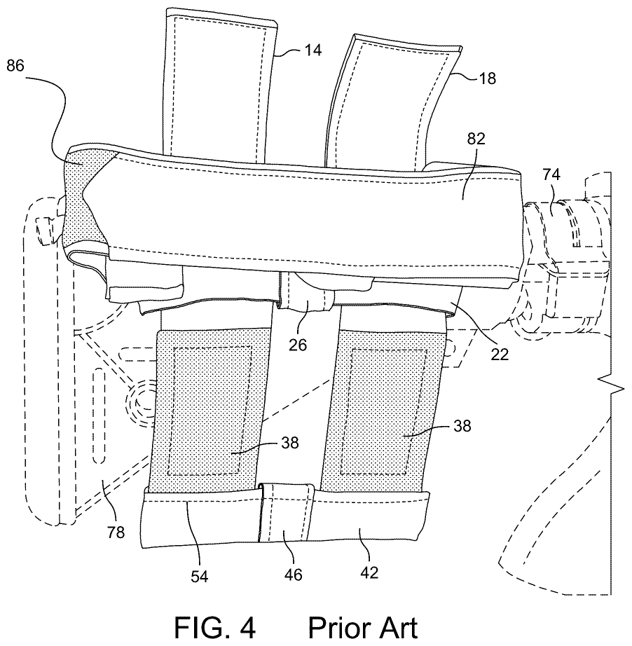

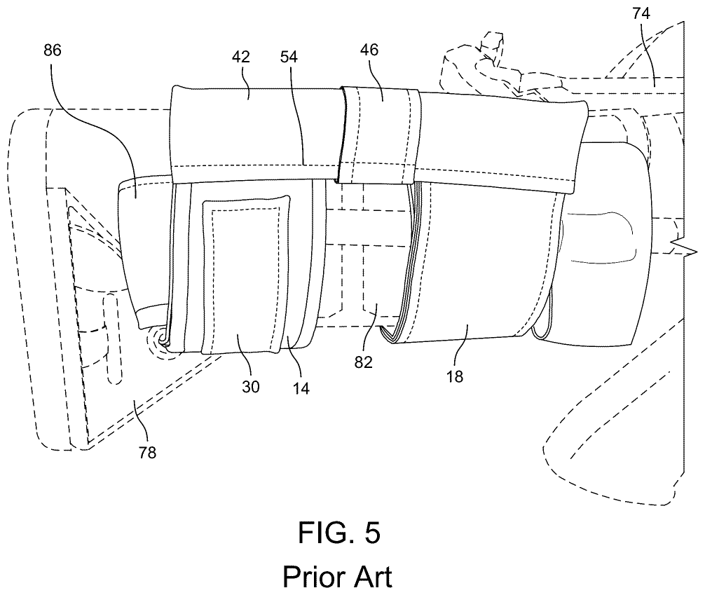

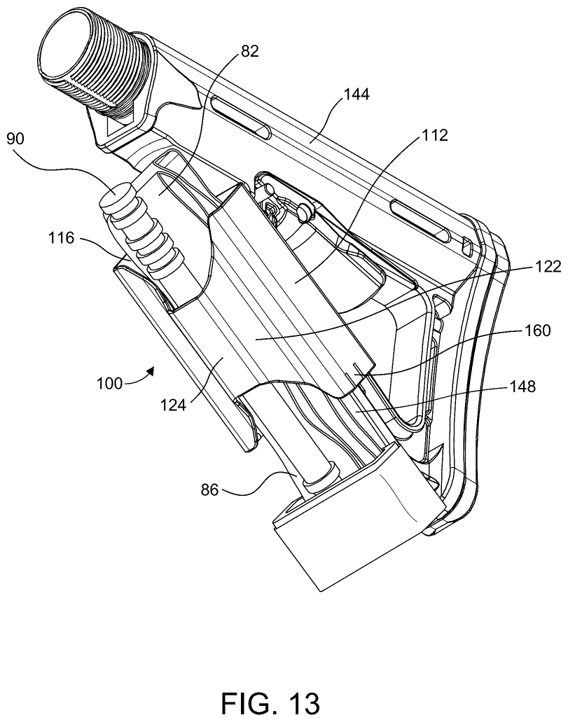

Referring now to FIGS. 1-5, there is shown a prior art tourniquet holster 10 for retaining a conventional tourniquet 82. The conventional tourniquet 82 consists of a hook-and-loop fastener belt 86 attached to an elongated windlass handle 90 and is among the most widely used tourniquets. The elongated windlass handle 90 (best illustrated in FIG. 13) is arranged to tighten the fastener belt 86 when rotated. As depicted, the tourniquet holster 10 includes a rearward elongate strap 14, a forward elongate strap 18, an end-support 42, and a cross-support 22. The end-support 42 is affixed, e.g., sewn, at one end of the straps 14, 18, and the cross-support 22 is affixed, e.g., sewn, at a positioned approximately midway along the length of the straps 14, 18. The end-support 42 and cross-support 22 maintain the straps 14, 18 in a generally parallel relationship to one another and at a predetermined distance from one another. Each strap 14, 18 is an elongate member sufficient in length to loop around a tourniquet 82 and fasten to itself utilizing suitable fasteners, e.g., hook-and-loop fasteners 38. Referring now to FIGS. 4 and 5, the dimensions of the straps 14, 18 are generally defined based on the size of the tourniquet 82. That is, the straps 14, 18 are designed to be sufficient in length to loop around and encircle the tourniquet 82 and overlap onto themselves. The straps 14, 18 are sufficient in width to substantially contain the tourniquet 82 therein, and sufficient in thickness to carry the tourniquet 82 during use.

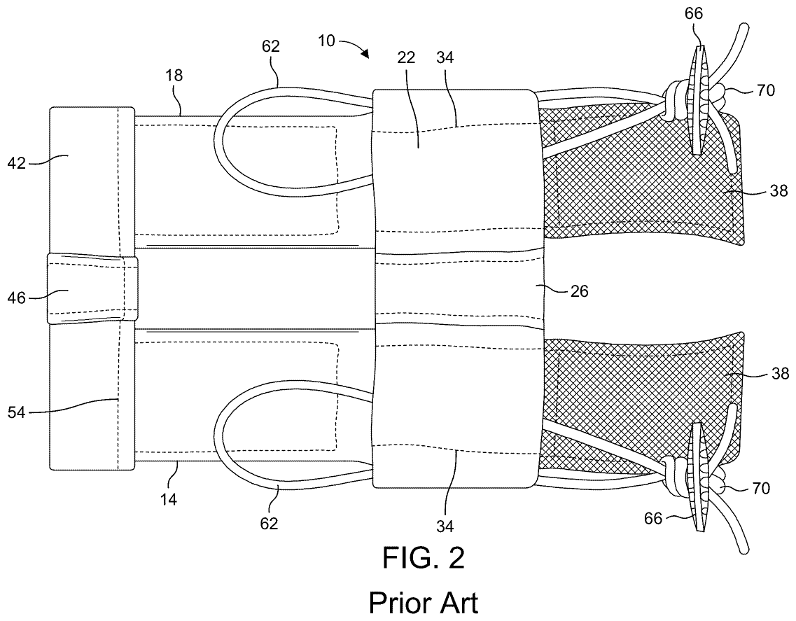

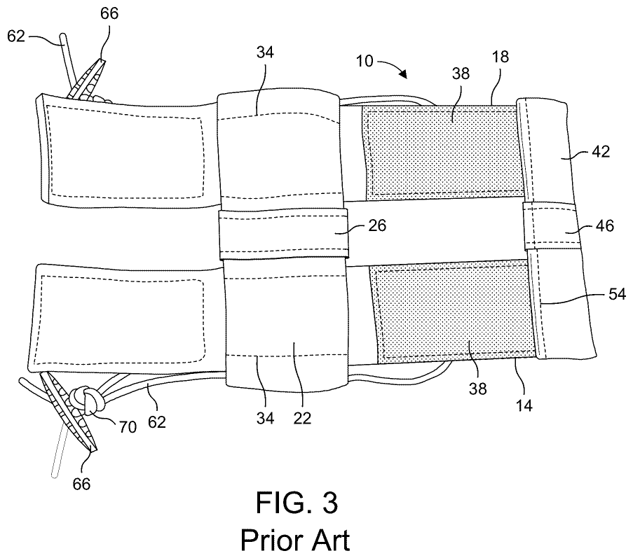

As best shown in FIGS. 2 and 3, an additional piece of fabric 46 may be affixed, e.g., sewn, to the end-support 42 in a position between the straps 14, 18 to serve as a stiffener for the end-support 42 to maintain the straps 14, 18 separated the predetermined distance. Similarly, an additional piece of fabric 26 may be affixed, e.g., sewn, to the cross-support 22 to serve the same purpose. The hook-and-loop fasteners 38 may be provided on the front and rear surfaces of the straps 14, 18, as indicated in FIGS. 2 and 3 to enable the straps to be fastened to themselves once they have been wrapped around the tourniquet 82 contained therein. Non-limiting examples of hook-and-loop-fastener material is Velcro®.

The tourniquet holster 10 is provided with bungee cords 62, each bungee cord 62 formed in a loop and threaded through an opening located at opposite ends of the cross-support 22. The bungee cords 62 are tied off at a knot 70 and each includes a disk or ball 66. The bungee cords 62 have the ability to stretch to provide tension to hold the tourniquet holster 10 in place against either side of a stock 78 of a rifle 74. As best seen in FIGS. 4 and 5, each bungee cord 62 may be threaded through a slot (not shown) in the stock 78 of the rifle 74 and have the ability to stretch to provide tension to hold the tourniquet holster 10 in place against either side of the stock 78 utilizing the disk or ball 66 in known ways. As discussed above, during use, when shifting the rifle stock from one shoulder to the other, it becomes necessary to adjust the tourniquet holster 10 from one side of the rifle stock to the other for the user to maintain the user's cheek firmly resting upon the rifle stock for stability and to avoid an obstructed view. As discussed above, this additional step costs valuable time, and under stress, can prove difficult to complete.

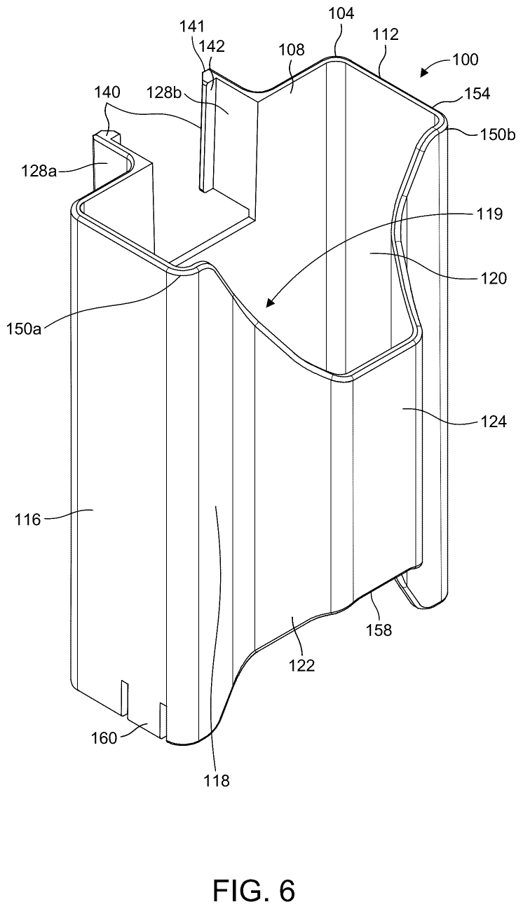

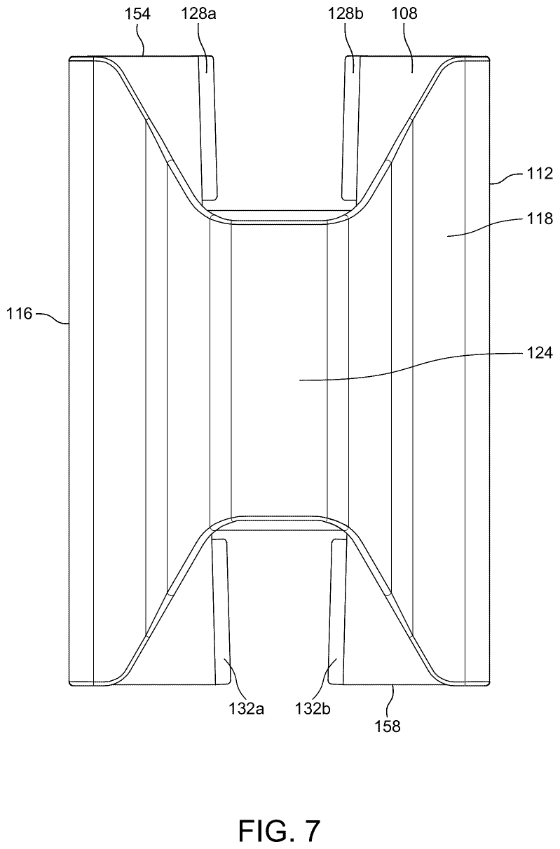

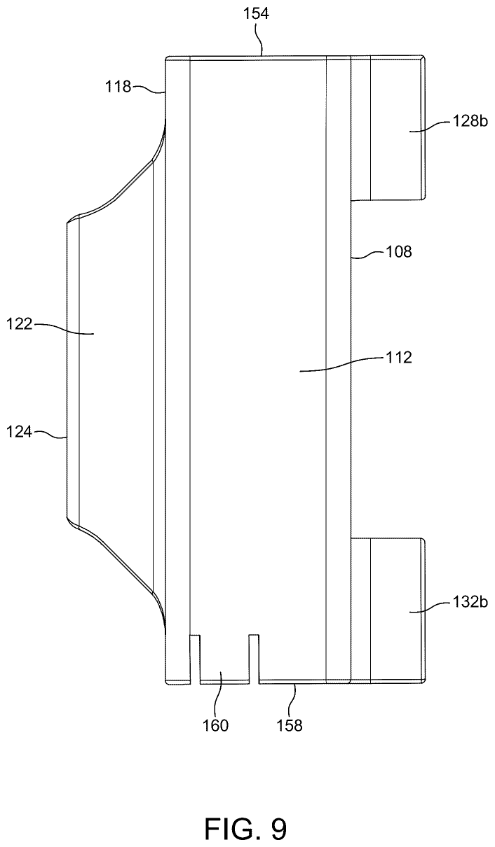

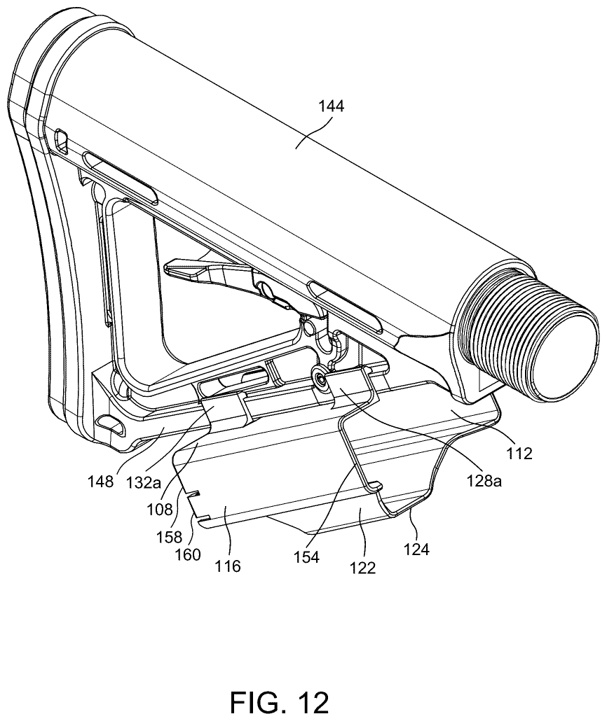

Referring now to FIGS. 6 through 13, there is shown a preferred embodiment 100 of the tourniquet holster of the present invention. In one embodiment, the tourniquet holster 100 is arranged to clip to a supporting article such as a rifle stock 144 utilizing an engagement mechanism comprising a plurality of retainer clips 128a, 128b, 132a, and 132b (shown in FIGS. 6 and 7) that extend laterally from the rear wall 108 of the holster 100. An exemplary rifle stock is the stock of an AR-15 rifle, which as illustrated in FIGS. 12 and 13, includes a diagonally extending brace member 148. As best shown in FIG. 6, each retainer clip includes an inwardly extending engagement tab 140 having an inclined surface 141 and an engagement surface 142. As the retainer clips are brought into contact with the brace member 148, the inclined surfaces 141 “ride up” the brace member 148 causing the retainer clips to spread apart from their original position to create spring tension. Once the engagement surfaces 142 clear the brace member 148, the retainer clips snap back to their original position to engage the brace member 148 within the retainer clips. In this manner, the holster 100 may be attached to the supporting article such as the rifle stock 144 without the need for special tools. It should be understood that the rifle stock 144 described herein and illustrated in the drawings is merely exemplary and it should be understood that the engagement mechanism of the holster may be modified as required to conform to the shape of other supporting articles including other types of rifle stocks to enable engagement therewith.

In one embodiment, the tourniquet holster 100 may include a rigid housing 104 of integral construction that is generally rectangular in shape, with the exception of a protrusion area 120 for housing the elongated windlass handle 90 of the tourniquet 82, discussed further below. The housing 104 may be formed of any durable, lightweight material, such as a thermoplastic material including an acrylic-polyvinyl alloy, or any other suitable material that will shield the tourniquet 82 housed therein from environmental factors, e.g., dirt and contaminants, and ensure that the functionality of the tourniquet 82 is not compromised. In one embodiment, the housing 104 may include opposed right and left sidewalls 112, 116, a rear wall 108, and a front wall 118. Together, the walls 108, 112, 116, and 118 define a sleeve channel 119 complementarily configured with the width of the fastener belt 86 of the tourniquet 82 to enable the tourniquet to easily pass through and be snugly retained within the sleeve channel 119 in a stored position in a space efficient and secure manner. In an embodiment, the housing 104 may be sized to expose a portion of the tourniquet 82 outside forward and rearward openings 154, 158 of the housing 104 when the tourniquet 82 is stored therein. In this manner, a user may grip this exposed portion of the tourniquet 82 and remove the tourniquet 82 from the holster 100. The tourniquet 82 may be one commercially available tourniquet having particular dimensions. Alternatively, in other embodiments, the housing 104 may be sized and shaped to snugly retain another commercially available tourniquet having different dimensions.

In an embodiment, the walls 108, 112, and 116 may be equal in height, each having an upper edge that combine to define the forward opening 154 and a lower edge that combine to define the rearward opening 158, the forward and rearward openings 154, 158 facilitating insertion of the tourniquet 82 within the sleeve channel 119 of the housing 104. The front wall 118 extends only partially from corners 150a and 150b of the housing 104 to a point where the front wall 118 meets a protrusion area 120.

In an embodiment, the protrusion area 120 may include protrusion sidewalls 122 that extend forward from the front wall 118. The protrusion sidewalls 122 taper to and are integral with a protrusion front wall 124. In an embodiment, the protrusion front wall 124 may be shorter in height than the opposed right and left sidewalls 112, 116. The protrusion front wall 124 may be positioned midway along the height of the sidewalls 112, 116 to form access areas to enable a user, e.g., police or military personnel, to quickly and easily finger grab and remove a tourniquet 82 from the holster 100 with one hand and with relative ease in the event of an emergency. The protrusion area 120 may also serve as a channel for the elongated windlass handle 90 of the tourniquet 82 to pass therethrough. In this manner, the often cumbersome elongated windlass handle 90 may be elegantly stored and released within the protrusion area 120. As best seen in FIG. 13, the elongated windlass handle 90 may rest on the fastener belt 86 of the tourniquet 82 when stored within the protrusion area 120.

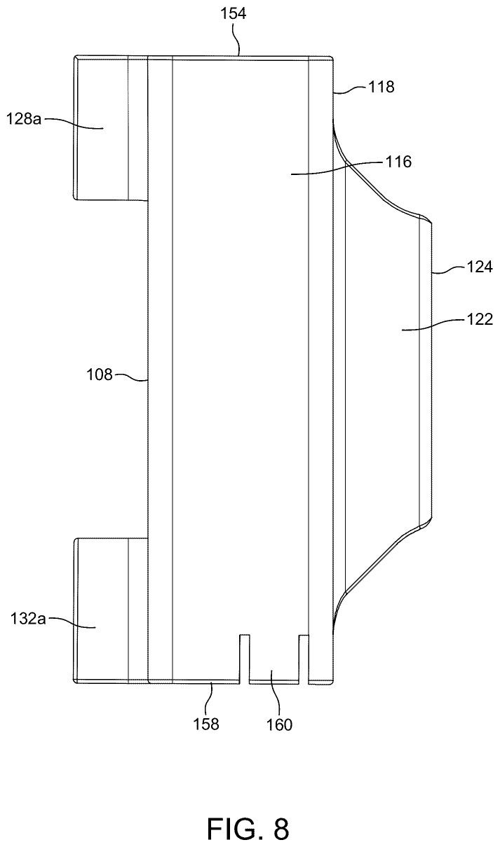

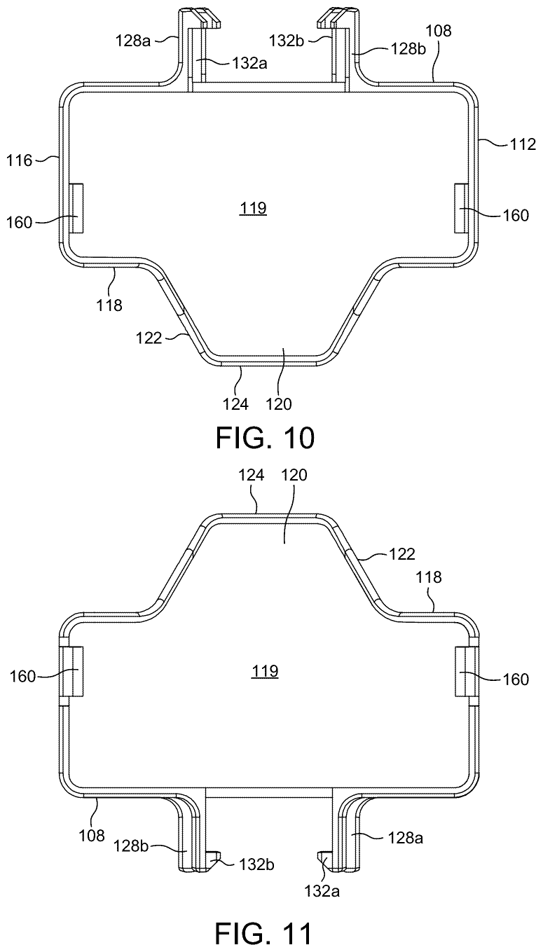

Referring now to FIGS. 8-11, each sidewall 112, 118 is provided with a retention tab 160 (FIG. 10) located proximate to the rearward opening 158. The retention tabs 160 are provided to restrict sliding movement of the tourniquet 82 and to retain the tourniquet 82 within the sleeve channel 119 during use of the rifle but to allow for easy removal of the tourniquet 82 from the sleeve channel 119 by a user in the event of an emergency. As best shown in FIGS. 10 and 11, the retention tabs 160 are rigid projections that project from the interior of the left and right sidewalls 112, 116 to prevent the tourniquet 82 from unintentionally sliding further than desired when the tourniquet 82 is received within the sleeve channel 119. This reduces the likelihood of the tourniquet 82 inadvertently exiting the holster 100 when stored.

Referring now to FIGS. 12 and 13, when attached to the brace member 148, the tourniquet holster 100 extends below the supporting article, e.g., the rifle stock 144, and out of the user's line-of-sight regardless of whether the rifle stock is positioned on the user's left or right shoulder. In this manner, when shifting the rifle stock from one shoulder to the other, there is no need to adjust the position of the holster 100 to eliminate an obstructed view, which may save valuable time, reduce injuries, and save lives. Additionally, there is no need to adjust the position of the holster 100 for the user to keep his cheek firmly pressed against the rifle stock to maintain stability. The holster 100 is retained in a position where the user can easily access the tourniquet 82.

During use, the tourniquet holster 100 is attached to the rifle stock 144 as described above with the tourniquet 82 inserted into the housing 104. During an emergency, a user can quickly access the tourniquet 82 and remove it from the holster 100, allowing the tourniquet 82 to be rapidly deployed for use.

The description herein is intended to illustrate possible implementations of the present invention and is not restrictive. While this disclosure has been made in connection with specific forms and embodiments thereof, it will be appreciated that various modifications other than those discussed above may be resorted to without departing from the spirit or scope of the claimed invention. Such variations, modifications, and alternatives will become apparent to the skilled artisan upon review of the disclosure. For example, functionally equivalent elements or method steps may be substituted for those specifically shown and described, and certain features may be used independently of other features, and in certain cases, particular locations of elements or sequence of method steps may be reversed or interposed, all without departing from the spirit or scope of the invention as defined herein. The scope of the claimed invention should therefore be determined with reference to the description above along with their full range of equivalents.

1. A tourniquet holster for accessibly supporting a tourniquet on a supporting article, the tourniquet including an elongated windlass handle, the holster comprising:
a. a rigid housing formed of opposed left and right sidewalls, a rear wall, and a front wall collectively defining a sleeve channel for retaining the tourniquet in a stored position therein;
b. protrusion sidewalls extending forwardly from the front wall and tapering to a protrusion front wall to collectively define a protrusion area for retaining the elongated windlass handle in a stored position therein; and,
c. the rigid housing sized to expose a portion of the tourniquet outside forward and rearward openings of the housing when the tourniquet is stored therein.

2. The tourniquet holster of claim 1, wherein the protrusion front wall is shorter in height than the left and right sidewalls to form access areas above and below the protrusion front wall.

3. The tourniquet holster of claim 1, additionally comprising a plurality of retainer clips extending laterally from the rear wall of the tourniquet holster to removably attach the tourniquet holster to the supporting article.

4. The tourniquet holster of claim 3, wherein each of the plurality of retainer clips includes an inwardly extending engagement tab formed of an inclined surface and an engagement surface.

5. The tourniquet holster of claim 1, wherein the supporting article is a rifle stock.

6. The tourniquet holster of claim 5, wherein when engaged to the rifle stock, the tourniquet holster extends below the rifle stock.

7. The tourniquet holster of claim 6, wherein the rifle stock is the stock of an AR-15 type rifle.

8. The tourniquet holster of claim 1, additionally comprising at least one rigid projection extending inwardly from the interior of the rigid housing to partially restrict the tourniquet from exiting the sleeve channel.

9. The tourniquet holster of claim 8, wherein the at least one rigid projection comprises a plurality of rigid projections extending inwardly from the opposed left and right sidewalls of the rigid housing.

10. The tourniquet holster of claim 1, wherein the elongated windlass handle defines a handle width and wherein the protrusion area defines a protrusion area width complimentarily configured with the handle width to enable the elongated windlass handle to pass through the protrusion area.

11. The tourniquet holster of claim 1, wherein the sleeve channel frictionally engages the tourniquet to inhibit the tourniquet from exiting the sleeve channel.

12. The tourniquet holster of claim 1, wherein the rigid housing is formed of a thermoplastic material.

13. The tourniquet holster of claim 1, wherein the exposed portion of the tourniquet outside the forward and rearward openings of the housing is large enough for a user to readily grip.

14. The tourniquet holster of claim 1, wherein the opposed left and right sidewalls, the rear wall, and the front wall are substantially flat surfaces.

15. A method for using a tourniquet holster comprising:
a. providing a tourniquet holster having
i. a rigid housing formed of opposed left and right sidewalls, a rear wall, and a front wall collectively defining a sleeve channel; and
ii. protrusion sidewalls extending forwardly from the front wall and tapering to a protrusion front wall to collectively define a protrusion area;
b. providing a rifle having a rifle stock;
c. providing a tourniquet including an elongated windlass handle;
d. engaging the tourniquet holster to the rifle stock;
e. retaining the tourniquet within the sleeve channel to expose a portion of the tourniquet outside forward and rearward openings of the sleeve channel; and,
f. retaining the elongated windlass handle within the protrusion area.
